(19) United States
(12) Patent Application Publication
(10) Pub. No.: US 2004/0225368 A1
(43) Pub. Date: Nov. 11, 2004

(54) Modular stud for posterior stabilised knee prosthesis

(76) Inventors: Sylvie Plumet, Chaumont (FR); Said Moussa, Chamarandes-Choignes (FR)

Correspondence Address: Edward G. Greive, Renner, Kenner, Greive, Bobak, Taylor & Weber, Fourth Floor, First National Tower, Akron, OH, 44308-1456, US

(21) Appl. No.: 10/829,726

(22) Filed: Apr. 22, 2004

(30) Foreign Application Priority Data
Apr. 24, 2003 (FR) 0305059

(51) Int. Cl. A61F 2/38

(52) U.S. Cl. 623/20.15; 623/20.32

(57) ABSTRACT

A tibial insert of a complete posterior stabilised knee prosthesis, noticeably of a trial prosthesis, has a base (5) on the upper part of which are formed one or several concave contact surfaces with one or several condyles of a femoral part, and a tibial stud (6) made to protrude, particularly perpendicularly from the base. The tibial stud is detachable from the base, and comprises a pin (12), coming from its lower surface, of a greater length than the thickness of the base. The base includes a bore (9) through which the tibial stud passes made to protrude below the lower surface than the base, opposite the upper surface.

This invention relates to a tibial stud of a knee prosthesis, posterior stabilised, particularly a trial knee prosthesis, as a knee prosthesis involving a stud of this type, particularly a trial prosthesis.

This type of prosthesis is made up of a tibial implant and a femoral implant. The tibial implant comprises a tibial disc on which a tibial insert is laid (also called a meniscus), generally in polyethylene. The tibial insert comprises on the one hand a base on the upper part of which the upper surfaces are formed, generally concave, designed to work with the external surfaces of the condyles, generally convex, of the femoral implant and on the other hand, a tibial stud which protrudes from the base, particularly perpendicularly, and designed to work with an associated femoral stud. The femoral stud is noticeably transversal as it extends from one condyle to the other of the femoral implant and buts up to the tibial stud to prevent an antero-posterior sliding by the femoral implant in the flexing area of the knee, in particular starting from a flex of 30°.

According to the anatomy of the femur and the tibia on which the prosthesis is to be fitted, particularly in relation to both their orientation and the volume of bony resection to be effected, it is necessary to select a tibial insert from several sizes of possible tibial inserts.

When the surgeon fits this type of prosthesis he must verify, on the one hand, the size of the implant and also the best position for the tibial stud. He must therefore have at his disposal a range of different possible tibial inserts, in the form of a trial prosthesis, which infers high levels of stock. In addition, it is necessary to have a stable trial prosthesis and one resistant to stress such as a real prosthesis, in order to be able to verify that the chosen size is adapted to the requirements.

This invention envisages a complete posterior stabilised knee prosthesis, particularly a trial prosthesis, which is made up of a tibial part which has a tibial disc, a femoral part and a tibial insert interposed between the femoral and tibial parts, fixed by means of a fixing method to the tibial plate and made up of a base on the upper part of which are formed one or several concave surfaces of contact with one or several condyles of the femoral part, and a tibial stud which protrudes, particularly perpendicularly, from the base, the tibial stud being assembled so that it is removable from the base, characterised in that the tibial stud is made up of a pin coming from the lower surface, its length being greater than the thickness of the base and the base is made up of a cross bore in which the pin passes to make it protrude beyond the lower surface of the base, opposite to the upper surface, being arranged in such a way so that the stud is not able to pivot with respect to the base, round the pin's axis.

Thus, the stud, after having passed through the base, can also penetrate a bore formed in the tibial plate, to receive the anchoring pin in the tibia. Thus it is possible to have available several bases of different sizes, particularly of different thickness, all with the same cross bore to receive a similar pin. With a single stud several differently sized inserts for trial prosthesis which are however very robust vis-à-vis the stresses and constraints and which facilitate genuine trials to be carried out. In particular, the pin which passes through the base holds the stud fixed to the base well in position, without it being able to rock as a result of any impact of the femoral part on the stud.

According to a refinement of the invention, the tibial stud is made up of a noticeably flat and non-circular shaped base plinth, in particular substantially rectangular or square, from which the pin protrudes, whilst the tibial insert comprises a cavity on its upper surface of a size and shape complementary to the plinth in order to receive it whilst preventing it rotating round the pin's axis.

According to a preferred method, the pin is cylindrical, particularly circular, and the bore is complimentary to the pin.

According to a preferred method, an O-ring wedge is threaded onto the pin, in order to facilitate the pin being locked in the bore.

This invention also envisages an assembly of tibial inserts, particularly for a trial prosthesis consisting of several differently sized bases and a tibial stud according to the invention, the base all having the same bore, to receive the same cavity.

A trial knee prosthesis is now described according to one method of the invention, purely as an example, relating to the drawings, in which:

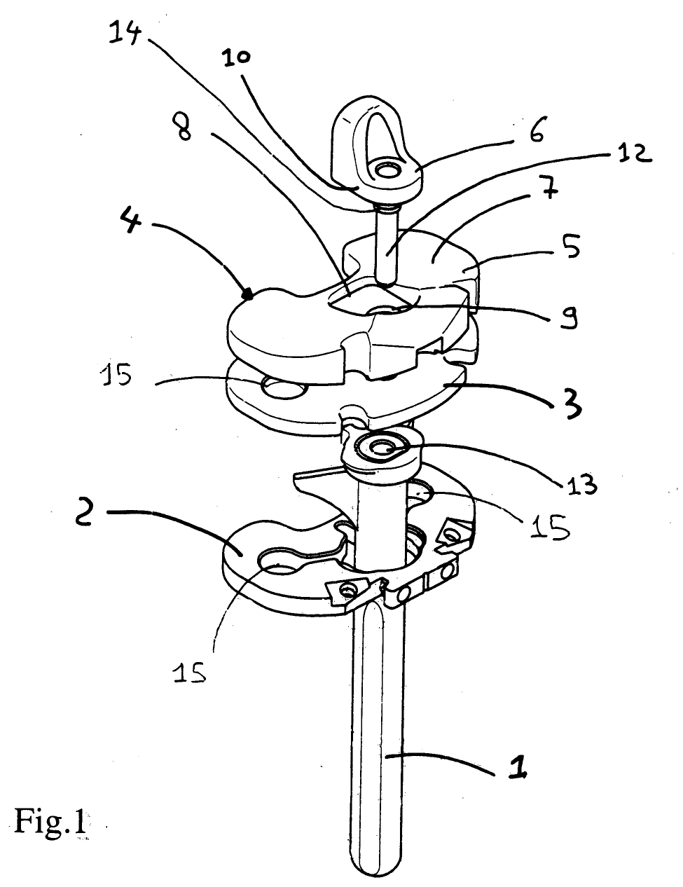

FIG. 1 represents a tibial insert fitted on a tibial part of a knee prosthesis, in its non-assembled state, and

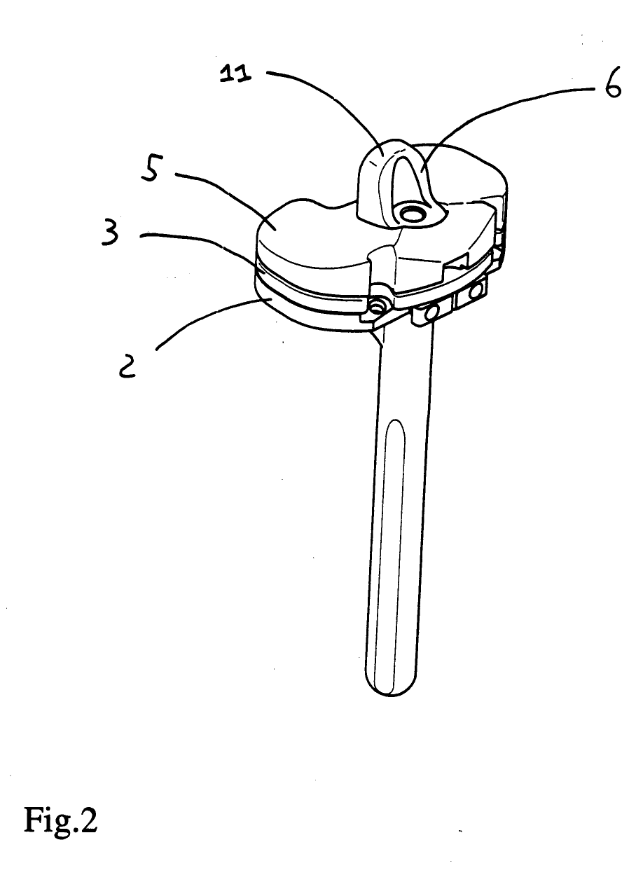

FIG. 2 represents the tibial part of FIG. 1, in its assembled state.

In FIG. 1, a tibial part of a trial knee prosthesis can be seen, comprising an anchorage pin 1 in the medullar channel of the tibia, a tibial disc 2, a cross-piece 3 and an insert 4, in polymer.

The insert 4 comprises a base 5 and a stud 6. The upper surface of the base 5 comprises concave contact surfaces 7 with the condyles of a femoral part.

The base 5 comprises a cavity 8 in the bottom, noticeably square in which a cross bore 9 is formed from the upper to the lower side of the base. The bore 9 has a circular section. The cross section of the bore is smaller than the bottom of the cavity 8.

Two fixing studs, left and right, are made to protrude below the base 5, and penetrate the complimentary left and right holes 15 formed in the tibial disc 2 and cross-piece 3, to allow the tibial insert 4 to be fixed to the disc 2. In particular, the insert 4 is prevented from rotating with respect to the disc 2 around the anchoring pin's 1 axis by these studs and complimentary shaped holes 15. Of course, other means of fixing the insert to the tibial disc are possible.

The stud 6 comprises a plinth 10 of a shape complementary to the cavity from which a sort of boss 11 comes upwards designed to make contact with a femoral stud stretching between the two condyles of the femoral part, in the medio-lateral direction.

The plinth 10 and the cavity 8 do not have a circular shape, so that the plinth cannot turn towards the interior of the cavity in relation to the anchoring pin's 1 axis.

A cylindrical and circular oblong pin 12 comes from the lower flat side of the stud 6 shaped complimentary to that of the bore 9.

When the prosthesis is assembled (FIG. 2), the stud penetrates the bore, fitted tightly, and the plinth 10 is positioned on the bottom of the cavity 8, in particular with a ratchet mechanism.

The length of the pin is greater than the thickness of the base at the level of the cross bore, so that the pin exits again from the other side.

The length of the pin is greater than the sum of the thickness of the base, of the thickness of the eventual cross-piece and of the thickness of the tibial plinth in such a way that it penetrates an anchoring pin cavity 13 formed in the anchoring pin of the prosthesis.

When, for example, there are four possible different thicknesses for the base, the pin is longer taking into consideration the greatest thickness.

An O-ring 14 is fitted around the pin, so that once it enters into the bore, the joint will be have a tendency to lock.

The fact that the pin passes through the base 5 and penetrates the anchoring pin facilitates a particularly rigid trial prosthesis and one resistant to stress, so that the trials that are carried out can be considered just as reliable as with a fixed stud prosthesis. Obviously, the advantage being that this reliability of the trial can be obtained without the need to have available a complete range of sizes of the tibial parts.

1. A posterior stabilised complete knee prosthesis, comprising
a tibial part which has a tibial disc, a femoral part and a tibial insert interposed between the femoral and tibial parts, fitted by fixing methods to the tibial disc and made up of a base on the upper part from which one or several concave contact surfaces are formed with one or several condyles of a femoral part, and
a tibial stud which protrudes, notably at right angles to the base, the tibial stud being detachable from the base, wherein
the tibial stud comprises a pin coming from its lower surface which is greater in length than the thickness of the base and
the base comprises a cross bore into which the tibial part goes so that it projects out of the lower surface of the base, opposite the upper surface, being constructed such that the stud cannot rotate in relation to the base round the pin's axis.

2. A prosthesis according to claim 1, wherein the tibial stud is made up of a noticeably flat, non-circular plinth, in particular noticeably rectangular or square, from which the pin protrudes, while the tibial insert comprises a cavity in its upper surface of a size and shape complementary to the plinth in order to prevent rotation round the pin's axis.

3. A prosthesis according to claim 1, wherein the pin is cylindrical in shape, noticeably circular, and the bore is of a shape complementary to the pin.

4. A prosthesis according to claim 1, wherein an O-ring wedge being threaded onto the pin, in order to facilitate it being locked in the bore.

5. A. prosthesis according to claim 1, in the form of a trial prosthesis.

6. A group of tibial inserts for trial prosthesis in accordance with claim 5, comprising
a plurality of bases of differing sizes, each of said bases having on its upper part one or more concave contact surfaces with one or more condyles of a femoral part;
a tibial stud having a pin coming from it's lower surface which is greater in length than the thickness of each base and
each base has a cross bore through which the tibial stud can pass in order to protrude out of the lower surface of each base, opposite the higher surface, being constructed in such a way that the stud cannot rotate in relation to each base and round the axis of the pin.

7. A group of tibial inserts according to claim 6, wherein the tibial stud comprises a base plinth noticeably flat and non-circular in shape, in particular substantially rectangular or square, with the pin projecting from it, while each base comprises a cavity in its upper surface of a dimension and shape complementary to the plinth in order to prevent rotation round the axis of the pin.

8. A group of tibial inserts according to claim 6, wherein the pin is cylindrical in shape, noticeably circular, and the bore is of a shape complementary to the pin.
